(19) United States
(12) Patent Application Publication
(10) Pub. No.: US 2009/0023993 A1
(43) Pub. Date: Jan. 22, 2009

(54) SYSTEM AND METHOD FOR COMBINED DISPLAY OF MEDICAL DEVICES

(76) Inventors: Tal Davidson, Yoqneam Illit (IL); Daphna Levy, Carmiel (IL); Kevin Rubey, Ventura, CA (US); Zvika Gilad, Haifa (IL); Jeremy Pinchas Gerber, Natanyah (IL); Michael Skala, Zichron Yaaqov (IL); Eli Horn, Kiryat Motzkin (IL)

Correspondence Address: Pearl Cohen Zedek Latzer, LLP, 1500 Broadway, 12th Floor, New York, NY, 10036, US

(21) Appl. No.: 12/175,819

(22) Filed: Jul. 18, 2008

Related U.S. Application Data
(60) Provisional application No. 60/929,921, filed on Jul. 18, 2007.

(30) Foreign Application Priority Data
Jul. 18, 2007 (JP) 2007-187159

(51) Int. Cl. A61B 1/04

(52) U.S. Cl. 600/109; 382/128

(57) ABSTRACT

A method and system to display combined information about an in-vivo lumen using several in vivo devices as data sources. A method is provided for interfacing different in vivo devices and viewing integrated results, on a combined display, by receiving in vivo data of at least two in vivo sensing procedures and analyzing the in vivo data to produce the combined representation. The combined representation may be displayed to a user during the course of an in vivo sensing procedure, and the in vivo data of an in vivo sensing procedure may be received and/or analyzed to produce a combined representation in real time.

This application claims the benefit under 35 U.S.C. § 119(e) of U.S. Provisional Patent Application No. 60/929,921, filed Jul. 18, 2007, which is hereby incorporated by reference in its entirety.

### FIELD OF THE INVENTION

The present invention relates to a system and method for presenting information of a body lumen provided by in vivo imaging devices.

### BACKGROUND OF THE INVENTION

Devices and methods for performing in-vivo imaging of passages or cavities within a body are known in the art. Such devices may include, inter alia, various endoscopic imaging systems and devices for performing imaging in various internal body cavities. Different devices, for example a capsule endoscope and a colonoscope, or a capsule endoscope and a double balloon endoscope, may provide different information of the same body lumen and may allow different functionalities. These devices each have a dedicated interface and display that are specialized per device's capabilities and method of operation. In some cases, a health-care professional may want to compare results from one procedure with results from a previous procedure. In other cases, the health-care professional may want to view results from previous procedures while performing a current procedure, or to provide specific controls or instructions in real time to an in vivo device based on previous findings from another device. Today the health-care professional can receive a video of in vivo images from a capsule, but may not be able to leverage it in order to find exactly where to reach with an endoscope for treatment.

Therefore there is a need in the art to provide a system and method for enabling a user to use results of different procedures and different devices in an enhanced approach.

### SUMMARY OF THE INVENTION

It is an object of the present invention to provide a combination of in vivo products using an integrated display which can provide more information to a user than each of the devices would provide when used independently.

According to one embodiment of the invention there is provided a method of interfacing different in vivo devices and viewing integrated results, on a combined display.

In another embodiment of the invention, a method is provided for displaying a combined representation to a user, for example by receiving in vivo data of at least two in vivo sensing procedures and analyzing the in vivo data to produce the combined representation.

According to one embodiment, a combined representation may be displayed to a user during the course of an in vivo sensing procedure. In some cases, receiving in vivo data of an in vivo sensing procedure and/or analyzing the in vivo data to produce a combined representation may be done in real time.

### BRIEF DESCRIPTION OF THE DRAWINGS

The present invention will be understood and appreciated more fully from the following detailed description taken in conjunction with the drawings in which:

FIG. 1 shows a schematic diagram of an in vivo imaging system according to one embodiment of the present invention;

FIG. 2 shows a representation of a combined user display according to one embodiment of the present invention;

FIG. 3 shows a representation of a combined user display according to another embodiment of the present invention; and

FIG. 4 is a flow chart showing a method for combining multiple in vivo sensing procedures and displaying an integrated result according to an embodiment of the invention.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

Embodiments of the system and method of the present invention are typically used in conjunction with an in-vivo sensing system or device. Examples of in-vivo sensing devices providing image data are provided in embodiments described in U.S. Pat. No. 7,009,634 to Iddan et al., which is hereby incorporated by reference in its entirety. Typically, a device according to the present invention includes video imaging capability, although it is within the scope of the present invention to include other types of imaging capabilities. In addition, the system and method according to the present invention may be used with any device, system and method sensing a body lumen or cavity.

While one typical use of embodiments of the present invention is imaging or examining the GI tract, other lumens may be imaged or examined.

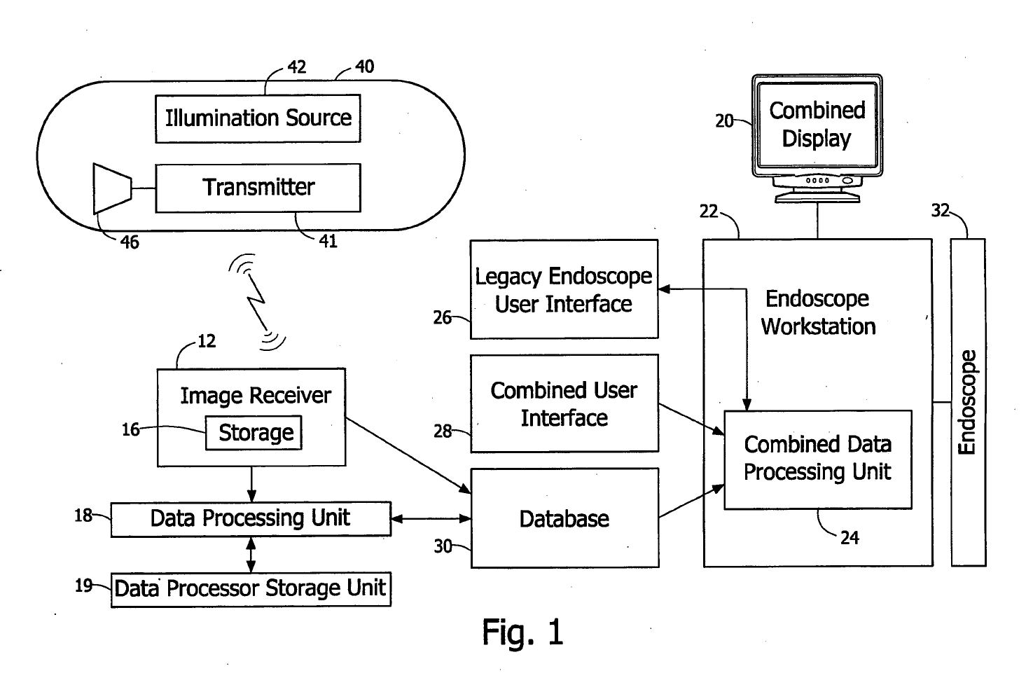

Reference is made to FIG. 1, which shows a schematic diagram of two in vivo imaging systems according to one embodiment of the present invention.

In an exemplary embodiment, the system may include an in vivo device 40, for example a capsule or other suitable device, having an imager 46, for capturing images, an illumination source 42, for illuminating the body lumen, and a transmitter 41, for transmitting and/or receiving data such as images and possibly other information to or from a receiving device. Preferably, the imager 46 is a suitable CMOS camera such as a “camera on a chip” type CMOS imager. In alternate embodiments, the imager 46 may be another device, for example, a CCD. According to some embodiments a 320×320 pixel imager may be used. Pixel size may be between 5 to 6 micron. According to some embodiments pixels may be each fitted with a micro lens. The illumination source 42 may be, for example, one or more light emitting diodes, or another suitable light source.

In alternate embodiments device 40 may be other than a capsule; for example, device 40 may be an endoscope, or other in vivo imaging device. An optical system, including, for example, a lens or plurality of lenses, may aid in focusing reflected light onto the imager 46. The device 40 may be inserted into a patient by for example swallowing and preferably traverses the patient's GI tract. In certain embodiments, the device and image capture system may be similar to embodiments described in U.S. Pat. No. 7,009,634 to Iddan et al. In alternate embodiments, other image capture devices, having other configurations, and other image capture systems, having other configurations, may be used.

Preferably, the in vivo imaging system collects a series of still images as it traverses the GI tract. The images may be later presented as, for example, a stream of images or a moving image of the traverse of the GI tract. The in vivo imager system may collect a large volume of data, as the in vivo device 40 may take several hours to traverse the GI tract, and may record images at a rate of, for example, two-eight images every second, resulting in the recordation of thousands of images. The image recordation rate (or frame capture rate) may be varied.

Preferably, located outside the patient's body in one or more locations, are an image receiver 12, preferably including an antenna or antenna array, an image receiver storage unit 16, a data processing unit 18 for processing and analyzing the image stream received by image receiver 12 and a data processor storage unit 19, for storing, inter alia, the images recorded by the device 40 and other information. Preferably, the image receiver 12 and image receiver storage unit 16 are small and portable, and may be worn on the patient's body during receiving and recording of the images. Data processor 18 and data processor storage unit 19 may be part of a personal computer or workstation which may include components such as data processor 18, a memory, a disk drive, and input-output devices, although alternate configurations are possible, and the system and method of the present invention may be implemented on various suitable computing systems. Data processor 18 may process raw image data received from receiver storage unit 16, to create videos, reports, and other data related to the in vivo procedure. Processed data may be transferred to a database 30. While the above example refers mainly to capsule-type endoscope, other examples of in vivo sensing devices may be used according to embodiments of the present inventions, such as double balloon endoscopes, colonoscopes, and gastro endoscopes.

Database 30 may include a storage unit, and may store medical data such as patient information, in vivo images, findings, patient history, procedure notes, etc. Database 30 may be included in an endoscope workstation 22, and may be located in other locations, for example, database 30 may be remote or accessed via a network such as the Internet. Database 30 may store general information such as pathologies database, or patient-specific information such as image data, patient history, video files, findings, etc.

Data processor 18 may include any suitable data processor, such as a microprocessor, multiprocessor, accelerator board, or any other serial or parallel high performance data processor. According to other embodiments a data processor may be included in image receiver 12 and images or other data may be displayed on a screen or display (not shown) on image receiver 12.

In operation, imager 46 may capture images and may send data representing the images to transmitter 41, which may transmit images to image receiver 12 using, for example, radio frequencies. Image receiver 12 may transfer the image data to image receiver storage unit 16. According to one embodiment, after a certain time of data collection, the image data stored in storage unit 16 may be sent to the data processor 18 or the data processor storage unit 19. For example, the image receiver storage unit 16 may be taken off the patient's body and connected to a personal computer or workstation which includes the data processor 18 and data processor storage unit 19 via a standard data link, e.g., a serial or parallel interface of known construction. The image data may be then transferred from the image receiver storage unit 16 to the data processor storage unit 19. Data processor 18 may analyze the data and provide the analyzed data to the database 30. In addition, the analyzed data may be presented on a monitor (not shown), where a health professional may view the image data. According to some embodiments the processing and/or displaying of images may be done on the image receiver 12.

Data processor 18 may operate software which, in conjunction with operating software such as an operating system and device drivers, may control the operation of data processor 18. Preferably, the software controlling data processor 18 includes code written in the C++ language and possibly additional languages, but may be implemented in a variety of known methods. According to some embodiments intermediate storage 16 need not be used.

The database 30 which may be included in endoscope workstation 22 may be contained within for example a computer readable storage medium, such as, but is not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storage. The database 30 may contain information related to each image, for example, scoring results, scoring formulas, text information, keywords, descriptions, a complete medical diagnosis, relevant cases, articles or images, for example, images of the close areas, images of pathology or any other information. In some embodiments, patients' details and history of a previous procedure or a plurality of previous procedures may be stored in the joint database 30 and may include different types of endoscopic procedures. A plurality of modalities may be used to obtain in vivo information, which may be stored in database 30. Other information, such as atlas images of known pathology types, may also be stored in database 30.

The image data collected and stored may be stored indefinitely, transferred to other locations or devices, manipulated or analyzed. According to some embodiments image data is not viewed in real time, other configurations allow for real time viewing.

The combined display 20 may present image data, combined from several in vivo imaging devices, preferably in the form of still and/or moving pictures, and in addition may present other information. In an exemplary embodiment, such additional information may include, but is not limited to a time line to show the time elapsed for each image, images in which a pathology, such as bleeding, had been identified by analysis of data of at least one of the in vivo devices, the location of a in vivo device in the patient's abdomen, etc. In an exemplary embodiment, the various categories of information are displayed in windows. According to some embodiments information that can aid a user in preparing a medical report may be displayed to the user while he is preparing a report. For example, a dictionary option may be presented so that the user may choose an appropriate term from a list of terms saved on the dictionary. An image database may be used to compare prior images to presently reviewed images, etc. Multiple monitors may be used to display image and other data.

Combined data processing unit 24 may be located in the endoscope workstation 22, or may be located externally to the endoscope workstation 22, or be remotely located. The combined data processing unit 24 may access database 30 and retrieve information related to previous procedures and to a current endoscopic procedure, which may be performed using endoscope 32. Based on analysis of the retrieved data, combined data processing unit 24 may produce an integrated analysis that may be presented to a user on combined display 20, allowing the user to control and operate it through a combined user interface 28. Examples of integrated analysis may include, but are not limited to, adding information from one modality to the native display of another modality, providing useful information, such as pathology location information, identified by one of the modalities during another procedure with the same or a different in vivo device, producing combined reports, findings, or recommendations for future treatment, etc. In some embodiments, information can be provided in a previous procedure's findings, for example findings from a capsule procedure, relating to recommended operation of a next procedure, for example an endoscopic procedure or a double balloon procedure. For example, a recommendation may be connected to a specific thumbnail of interest that had been detected by the capsule procedure. In some embodiments, it may be useful to provide a graph of changes or progress compared to previous procedures, such as changes in the amount of pathologies found, growth progress in the size of detected pathologies, or other changes relating to parameters detected in the procedures based on the image streams.

In some embodiments, obscure findings of one procedure may be more comprehensive by enhanced or augmented data from another procedure. In other cases, findings in one procedure may be contradicted or opposed by findings from another procedure. According to one embodiment, a location can be marked during one procedure, for example, by injecting fluorescent compound to the tissue during an endoscopic procedure, and the next procedure will be able to clearly identify the marked location for further or updated diagnosis.

According to one embodiment, an endoscope may insert a capsule during the same procedure, in order to create correlated image stream examples between different modalities. Such correlated image streams may be used by image processing learning algorithms to automatically correlate image streams obtained through different modalities. In other embodiments, correlation of the image streams may be performed by identifying a number of similar images throughout the streams, either manually by a health care professional or automatically by image processing. According to one embodiment, the image colors of the different in vivo imaging devices are correlated for display in order to allow easy comparison between parallel images of different in vivo imaging devices. According to one embodiment, the health care professional may manually perform the comparison. According to another embodiment, combined data processing unit 24 may automatically perform the comparison.

In one embodiment, the combined user interface 28 may be organized and managed by a medical case management tool, which may be located on the workstation or accessed remotely, for example through a local network or through the Internet. Combined user interface 28 may operate controlling software that manages both capsule endoscopy data and other types of endoscopic procedure related data, such as double balloon or colonoscopy data. Combined data processing unit 24 may allow working in a “legacy” mode, which represents only the standard legacy display of endoscope workstation 22 and operating options to a user through the legacy endoscope user interface 26. Another legacy mode could allow the user to interface the legacy capsule endoscopy analysis software. The legacy endoscope user interface 26 represents the current state of the art, in any single type of in vivo imaging device. According to one embodiment, two or more legacy displays are provided to a user, and importing information such as thumbnails from one of the modalities to the other may be performed by simple drag-and-drop operation. In a preferred embodiment of the present invention, combined data processing unit 24 may allow an advanced mode which will allow presentation and operation of the combined features of an endoscope workstation 22 and data from other in vivo imaging devices. Such data may be available on database 30 or accessed remotely, and may be analyzed by combined data processing unit 24. In some embodiments, the combined display of two different modalities is performed; however the present invention may be implemented by combining a plurality of modalities.

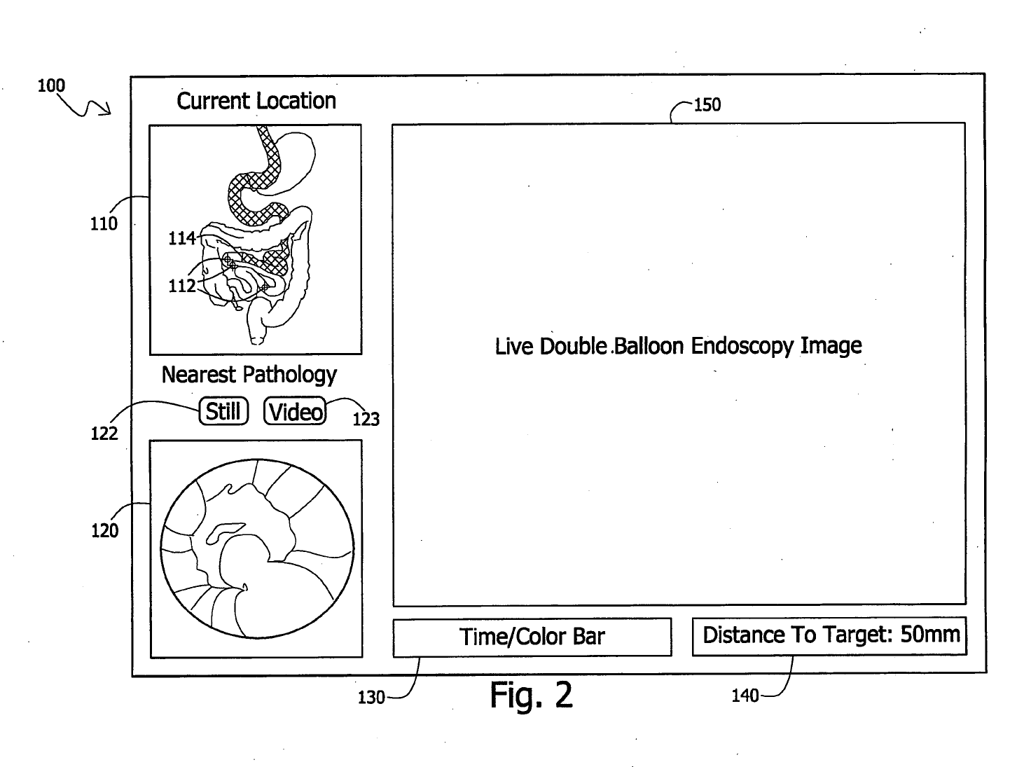

Reference is made to FIG. 2, which shows a representation of a combined user display and combined user interface of a capsule endoscopy procedure and a double balloon endoscopy system according to one embodiment of the present invention.

In one example, a capsule endoscopy procedure is performed initially to receive information about pathologies in a specific patient, and the analyzed data from the procedure is used during another procedure, for example a double balloon endoscopy procedure which is performed as a complementary treatment or diagnosis procedure. During a double balloon procedure, the health care professional may want an alert that a previously identified pathology has been reached. In another embodiment, after a capsule endoscopy procedure, the health care professional may automatically receive suggested methods of treatment. For example, the pre-test findings of a capsule endoscopy may include a recommendation of how to perform the treatment, for example, from where to enter with a double balloon endoscope, and may be presented (for example on combined display 20) to the health care professional automatically prior to starting the double balloon procedure. Once the double balloon procedure is in process, real-time analysis of the current double balloon image may be performed and compared to the capsule images of a previous procedure, to provide an estimated location of the double balloon endoscope on a schematic diagram of the treated body lumen, for example as shown in window 110. The treated body lumen may be displayed to a user, for example by presenting a schematic diagram of the region of interest, and the current estimated location reached by the double balloon 114 may be marked or highlighted on the diagram. The location of a polyp may be pointed out to the health care professional in several methods, such as marking the estimated location on the combined display 20 (for example marked location 112), providing audio alerts when the endoscope is near the location, providing an estimated distance to target location (shown in window 140), etc. Methods for calculating the distance to a target location in a body lumen may be similar to those described in US Patent Application Publication Number 2006/0036166, entitled: “SYSTEM AND METHOD FOR DETERMINING PATH LENGTHS THROUGH A BODY LUMEN”. If several pathologies were found as a result of the capsule endoscopy procedure analysis, all of them may be highlighted on the combined display 20 (marked locations 112), or only the nearest ones may be highlighted. A still image of the pathology from the previous procedure may be displayed to the user in a small window (shown in window 120) to allow easy identification of the pathology during the current procedure. According to some embodiments, a small window overlapping the main view of the current procedure may show the pathologies found in the previous procedures, in order not to interfere substantially with the main view of the current procedure.

The health care professional may be able to press a button on a keyboard, on a touchscreen on the workstation display or on the endoscope's handset in order to play a short clip of the areas just before or just after a pathology (for example, buttons 122 and 123). In one embodiment, three pathologies may be automatically displayed on the combined display 20 as the health care professional advances with, for example, the double balloon endoscope: a previous thumbnail, and the next two thumbnails. According to another embodiment, manual selection, for example through the handset of the double balloon endoscope, is enabled. In one example, the user will be able to activate specific modes of operation, such as activate a view of narrow band imaging of the currently viewed region, at the click of a new button added to the handset of the endoscope workstation 22. A time/color bar (window 130) may be presented to the user to indicate the imaging time scale and image color scale of the obtained images during the current procedure or as cross reference to the previous capsule procedure. According to one embodiment, a toggle button may enable selecting between different optional time/color bars.

Additional buttons on the endoscope handset may be used to activate the combined user interface. If the health care professional's hands are occupied, additional foot pedals may also be used to activate certain features of the combined user interface, once again providing the benefit of not requiring any hand motion, and enabling the health care professional to focus on the endoscope procedure. For example, the health care professional will be able to view the nearest detected pathology as provided based on the previously performed capsule endoscopy procedure, by pressing the foot pedal. In other embodiments, the target image may appear on the combined display automatically, only when the double balloon or other endoscope reaches the vicinity of the target. According to one embodiment, selected thumbnails are displayed to a user on the combined display 20 in the correct order of the current procedure. If both capsule endoscopy and double balloon endoscopy procedures start from mouth, the detected and/or selected images are displayed to the user in the same order they were obtained. If capsule endoscopy starts from mouth but current double balloon or other endoscopy procedure starts from anus, the capsule endoscopy selected thumbnails will be displayed in last-in-first-out (LIFO) order so the user will view them as they appear in the current double balloon endoscopy procedure. The displayed thumbnails can be selected manually or automatically. In some embodiments, the health care professional may also provide voice commands, which may be interpreted to the combined user interface 28 by known speech recognition methods. When using voice commands, the user will not need to release the endoscope handset at all and may receive the same functionality as describe above without pressing any buttons.

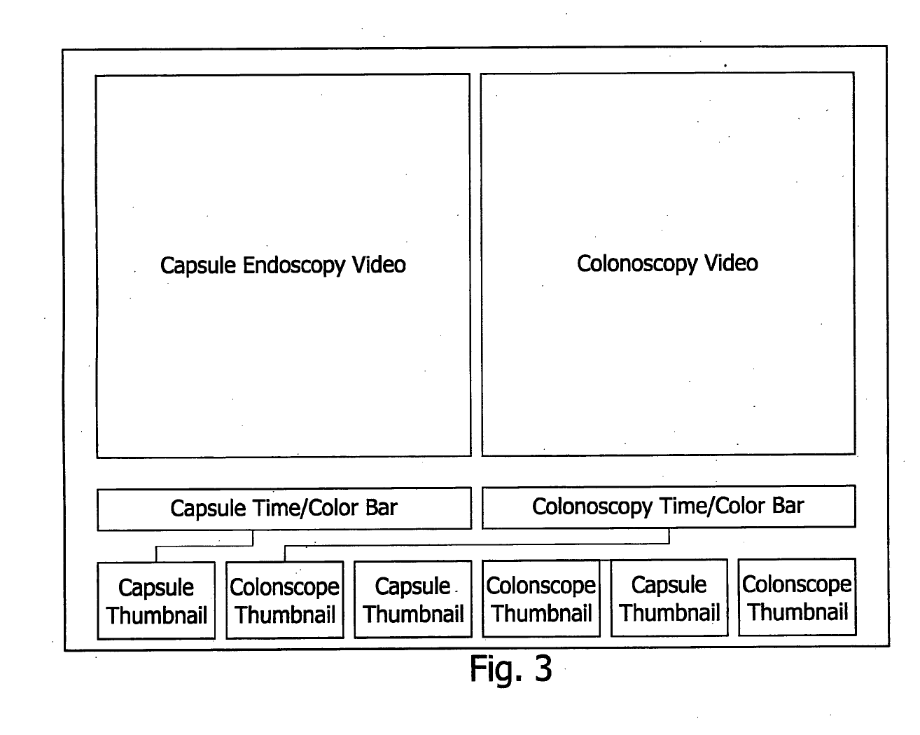

Reference is made to FIG. 3, which shows a representation of a combined user display according to another embodiment of the present invention. In this typical embodiment, a specific patient had undergone two different procedures for imaging the GI tract, a capsule endoscopy procedure and a colonoscopy procedure. A health care professional may want to review the results that were produced by both procedures. In some cases, a health care professional may want to compare certain selected images from one procedure with parallel images from another procedure. According to one embodiment, the user may want to manually select interesting images or short clips from both procedures for comparison. In another embodiment, the user may request to display automatically identified matching sections of the procedures. For comparison and processing purposes, it may be useful to store the complete image stream of all performed procedures, regardless of their type, for example storing capsule image streams, endoscope image streams etc. These streams may be used for offline or online (e.g., real time or almost real time) comparison of one procedure to other procedures. In other embodiments, selected sections an image stream may be stored and used for processing. An indication of the time passed from the beginning of each procedure may also be useful when preparing a diagnosis. In some cases, a report may automatically be produced based on joint findings from two or more procedures, and may include selected images such as thumbnail images from two or more in vivo devices. Images from a pathology image atlas may be presented to the user for simplifying diagnosis in the combined display. The image atlas may be based on images obtained in several different modalities. According to one embodiment, the capsule atlas may be enhanced by adding correlating images of pathologies obtained through other modalities, for example an endoscope, and the recommended treatment may be presented to the user as well. By adding the recommended treatment, the capsule findings and/or reports and/or display may comprise a therapeutic element, such as the recommended procedure for therapy or the recommended procedural element for therapy. According to one embodiment, the combined display may allow cross reference of findings, notes, selected images, movie clips, etc, between different procedures performed on the same patient or on different patients. For example, the combined display may allow exporting movie clips, such as interesting clips containing images of pathologies found, from one procedure to another. According to one embodiment of the present invention, reports may be created by dragging-and-dropping objects from one legacy display to another, or from a combined display to a tool that can create joint reports. A joint report may include selected images from any of the in vivo imaging devices available for a specific patient. According to one embodiment, the joint report may optionally be produced by the same tool used for creating a legacy capsule endoscopy report, for example.

In some embodiments, a capsule endoscopy procedure may be performed after a colonoscopy procedure. For example, colonoscopy may be performed on a patient in order to remove a large-sized polyp, for example a polyp of 5 mm in length. A health care professional may want to perform a check after some time that the treated area healed properly. In another example, the 5 mm-polyp may not be removed, and a capsule endoscopy may be performed after a certain time period, for example one year later, to check the current size of the polyp. In such cases, the combined display may provide the differences in measured sizes of polyps found in one procedure, compared to the measured sizes of polyps found in the next procedure. In one embodiment, a previous colonoscopy procedure can provide operation information to a current capsule endoscopy procedure. For example, if the health care professional performs a capsule endoscopy procedure after the removal of a polyp with a colonoscope, the capsule may be programmed with specific data such as the location of the surgery in the colon, in order to increase capsule frame rate at the area of the operation, to verify complete recovery. While viewing a capsule endoscopy analyzed video or online video, previous endoscopic procedure data may be used to alert a user that a pathology which was previously found is coming up. One or more time/color bars may be presented to the user to indicate the imaging time scale and image color scale of the obtained images during the current procedure, and/or previous procedures.

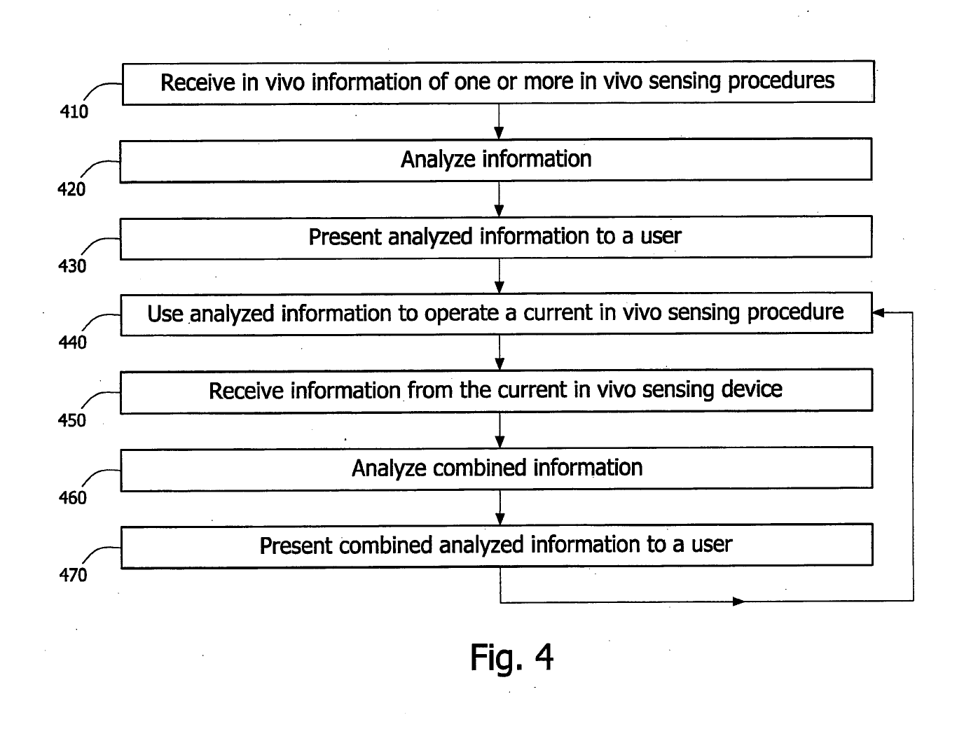

Reference is made to FIG. 4, which is a flow chart showing a method for multiple in vivo sensing procedures and displaying a combined result, according to an embodiment of the invention. In step 410, in vivo information of one or more in vivo sensing procedures is received, for example by a processor (such as combined data processing unit 24 described in FIG. 1). The in vivo information may be received directly from an in vivo device, and/or may be retrieved from a database or from a data storage unit. Typically, the in vivo sensing procedures provide image data of a previous in vivo sensing procedure. The image data is processed and analyzed (step 420). In some embodiments, the information may be analyzed remotely, by a remote system or processor. In some embodiments, more than one previous procedure is used, and the analysis may include combining information of a plurality of in vivo procedures. The analyzed information and/or combined information may be presented to a user (step 430). For example, an in vivo imaging capsule may have been swallowed by a patient some time ago to visualize the patient's esophagus. The analyzed information may be used to operate another in vivo sensing device (step 440). The data may have been analyzed on a remote system, and the analyzed data may have been imported to a database which may be used by the other in vivo sensing device. For example, the analyzed information may be used before, during or after performing an esophagus endoscopic procedure on the same patient. In step 450, information is received from the current in vivo sensing device, typically a different in vivo device than the in vivo device used in a previous procedure or procedures. The information from the current device may be received in real time, or in almost real time and accessed by the processor. The information may be analyzed (step 460) and combined with, for example, all available information relating to the same patient then may be presented to a user (step 470). The information may be combined and analyzed in real time or in almost real time, and provided to a health care professional as useful information during the current procedure. In one example, the analyzed information may include information regarding a target pathology which needs to be treated in the current procedure. For example, the analyzed information may provide the health care professional with an estimated or calculated distance to a previously identified pathology, using combined information from the current procedure, such as the current position of the in vivo sensing device in the patient's body and information from a previous procedure, such as the location and type of the pathology.

It will be appreciated by persons skilled in the art that the present invention is not limited by what has been particularly shown and described herein above. Rather, the scope of the invention is defined by the claims that follow:

1. A method for combined display of in vivo sensing procedures comprising:
receiving in vivo data of at least two in vivo sensing procedures, said data obtained using at least two different modalities;
analyzing said data to produce a combined display; and
displaying the combined display to a user.

2. The method of claim 1 wherein the combined display is displayed to a user during the course of an in vivo sensing procedure.

3. The method of claim 1 wherein receiving the data of at least one of the in vivo sensing procedures is in real time.

4. The method of claim 1 wherein one of the at least two in vivo sensing procedures is a capsule endoscope imaging procedure.

5. The method of claim 4 further comprising:
identifying a location of the capsule endoscope; and
displaying the identified location in the combined display.

6. The method of claim 1 further comprising:
identifying pathology information by analysis of said data; and
displaying the pathology information in the combined display.

7. The method of claim 1 further comprising: correlating in vivo data streams obtained using the at least two different modalities.

8. The method of claim 1 further comprising: providing a graph of changes to compare current and previous procedures.

9. The method of claim 1 further comprising: correlating image colors of the different modalities for the combined display.

10. The method of claim 1 further comprising: inserting a capsule endoscope during a double balloon endoscope procedure.

11. A system for displaying medical data combined from at least two in vivo modalities, comprising:
a combined data processing unit to combine data from said at least two in vivo modalities; and
a combined user interface to display said combined data.

12. The system of claim 11 wherein one of the at least two modalities comprises a capsule endoscope.

13. The system of claim 11 wherein one of the at least two modalities comprises a double balloon endoscope.

14. The system of claim 13, wherein the endoscope comprises additional buttons to activate the combined user interface.

15. The system of claim 11, further comprising an image processor to automatically correlate image streams from said at least two in vivo modalities; wherein said at least two in vivo modalities comprise imaging devices.

16. The system of claim 11, further comprising a medical case management tool to control the combined display.
